(19) United States
(12) Patent Application Publication
(10) Pub. No.: US 2010/0183629 A1
(43) Pub. Date: Jul. 22, 2010

(54) ANTAGONISTS OF ENDOTHELIAL DIFFERENTIATION GENE SUBFAMILY 3 (EDG-3, S1P3) RECEPTORS FOR PREVENTION AND TREATMENT OF OCULAR DISORDERS

(75) Inventors: Debra L. Fleenor, Crowley, TX (US); Allan R. Shepard, Fort Worth, TX (US); Iok-Hou Pang, Grand Prairie, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/749,331

(22) Filed: Mar. 29, 2010

Related U.S. Application Data
(63) Continuation of application No. 11/828,137, filed on Jul. 25, 2007.
(60) Provisional application No. 60/833,080, filed on Jul. 25, 2006.

(51) Int. Cl. A61K 39/395; A61K 38/02; A61K 31/426; A61K 31/54; A61K 31/661; A61K 31/167; A61K 31/17; A61P 9/12; A61P 27/02; A61P 27/06

(52) U.S. Cl. 424/172.1; 514/2; 514/365; 514/226.8; 514/119; 514/626; 514/597

(57) ABSTRACT

Antagonists of S1P3 (Edg-3) receptors are provided for attenuation of Smad signaling in a method of down-regulation of receptor signaling and downstream decreased production of connective tissue growth factor in ocular disorders involving CTGF accumulation. Ocular disorders involving inappropriate CTGF accumulation include ocular hypertension, glaucoma, glaucomatous retinopathy, optic neuropathy, macular degeneration, diabetic retinopathy, choroidal neovascularization, proliferative vitreoretinopathy and ocular wound healing, for example. Such disorders are treated by administering antagonists of the present invention.

### CROSS-REFERENCE TO RELATED APPLICATION

This application is a Continuation (CON) of co-pending U.S. application Ser. No. 11/828,137, filed Jul. 25, 2007, priority of which is claimed under 35 U.S.C. §120, the contents of which are incorporated herein by reference. This application also claims priority under 35 U.S.C. §119 to U.S. Provisional Patent Application No. 60/833,080, filed Jul. 25, 2006, the contents of which are incorporated herein by reference.

### TECHNICAL FIELD OF THE INVENTION

The present invention relates to the field of compositions for attenuation of endothelial differentiation gene subfamily 3 receptors for down-regulation of receptor signaling and downstream decreased production of connective tissue growth factor (CTGF) in ocular disorders involving CTGF accumulation.

### BACKGROUND OF THE INVENTION

Most ocular disorders are associated with cellular processes including cell proliferation, survival, migration, differentiation, and angiogenesis. CTGF is a secreted cytokine believed to be a central mediator in these cellular processes. In particular, CTGF is known to increase extracellular matrix production via increased deposition of collagen I and fibronectin. Overexpression of CTGF has been implicated as a major causative factor in conditions such as scleroderma, fibroproliferative diseases, and scarring in which there is an overaccumulation of extracellular matrix components.

An overaccumulation of extracellular matrix materials in the region of the trabecular meshwork (TM) is a hallmark of certain forms of glaucoma; such increases are believed to lead to increased resistance to aqueous outflow and, therefore, elevated intraocular pressure (IOP). International Patent Application No. PCT/US2003/012521 to Fleenor et al., published Nov. 13, 2003, as WO 03/092584 and assigned to Alcon, Inc. describes the elevated presence of CTGF mRNA in glaucomatous TM cells vs. normal TM cells. Thus, it is believed that CTGF plays a role in extracellular matrix production by the trabecular meshwork cells.

The trabecular meshwork (TM) is a complex tissue including endothelial cells, connective tissue, and extracellular matrix located at the angle between the cornea and iris that provides the normal resistance required to maintain a normal IOP. An adequate IOP is needed to maintain the shape of the eye and to provide a pressure gradient to allow for the flow of aqueous humor to the avascular cornea and lens. Excessive IOP, commonly present in glaucoma, has deleterious effects on the optic nerve, leads to loss of retinal ganglion cells and axons, and results in progressive visual loss and blindness if not treated. Glaucoma is one of the leading causes of blindness worldwide.

Primary glaucomas result from disturbances in the flow of aqueous humor that has an anatomical, biochemical or physiological basis. Secondary glaucomas occur as a result of injury or trauma to the eye or a preexisting disease. Primary open angle glaucoma (POAG), also known as chronic or simple glaucoma, represents ninety percent of all primary glaucomas in the United States. POAG is characterized by the pathological changes in the TM, resulting in abnormally high resistance to fluid drainage from the eye. A consequence of such resistance is an increase in the IOP.

Certain drugs such as prednisone, dexamethasone, and hydrocortisone are known to induce glaucoma by increasing IOP. Further, the mode of administration appears to affect IOP. For example, ophthalmic administration of dexamethasone leads to greater increases in IOP than does systemic administration. Glaucoma that results from the administration of steroids is termed steroid-induced glaucoma.

Current anti-glaucoma therapies lower IOP by the use of medications to suppress aqueous humor formation or to enhance aqueous outflow, as well as surgical procedures, such as laser trabeculoplasty, or trabeculectomy, to improve aqueous drainage. Pharmaceutical anti-glaucoma approaches have exhibited various undesirable side effects. For example, miotics such as pilocarpine can cause blurring of vision and other negative local side effects. Systemically administered carbonic anhydrase inhibitors can cause nausea, dyspepsia, fatigue, and metabolic acidosis. Further, certain beta-blockers have been associated with pulmonary side effects attributable to their effects on beta-2 receptors in pulmonary tissue. Alpha2-agonists can cause tachycardia, arrhythmia and hypertension. Such negative side effects may lead to decreased patient compliance or to termination of therapy.

U.S. Published Patent Application No. 2005/0234075 to Fleenor et al., published Oct. 20, 2005, hereby incorporated by reference herein, provides GSK-3 and CDK inhibitors having inhibitory activity for both basal and TGFβ2-induced CTGF expression in human trabecular meshwork cells.

Macular degeneration is the loss of photoreceptors in the portion of the central retina, termed the macula, responsible for high-acuity vision. Degeneration of the macula is associated with abnormal deposition of extracellular matrix components in the membrane between the retinal pigment epithelium and the vascular choroid. This debris-like material is termed drusen. Drusen is observed with a funduscopic eye examination. Normal eyes may have maculas free of drusen, yet drusen may be abundant in the retinal periphery. The presence of soft drusen in the macula, in the absence of any loss of macular vision, is considered an early stage of AMD.

Choroidal neovascularization commonly occurs in macular degeneration in addition to other ocular disorders and is associated with proliferation of choroidal endothelial cells, overproduction of extracellular matrix, and formation of a fibrovascular subretinal membrane. Retinal pigment epithelium cell proliferation and production of angiogenic factors appears to effect choroidal neovascularization.

Diabetic retinopathy is an ocular disorder that develops in diabetes due to thickening of capillary basement membranes and lack of contact between pericytes and endothelial cells of the capillaries. Loss of pericytes increases leakage of the capillaries and leads to breakdown of the blood-retina barrier.

Proliferative vitreoretinopathy is associated with cellular proliferation of cellular and fibrotic membranes within the vitreous membranes and on the surfaces of the retina. Retinal pigment epithelium cell proliferation and migration is common with this ocular disorder. The membranes associated with proliferative vitreoretinopathy contain extracellular matrix components such as collagen types I, II, and IV and fibronectin, and become progressively fibrotic.

Wound healing disorders may lead to severe ocular tissue damage via activation of inflammatory cells, release of growth factors and cytokines, proliferation and differentiation of ocular cells, increased capillary permeability, alterations in basement membrane matrix composition, increased deposition of extracellular matrix, fibrosis, neovascularization, and tissue remodeling.

In view of the importance of the above-cited ocular disorders, particularly the pathological damage to the trabecular meshwork and damage due to overproduction of extracellular matrix, it is desirable to have an improved method of treating these ocular disorders that addresses underlying causes of its progression.

Abbreviations as used herein include:
AC Adenylyl cyclase
AP-1 Activator protein 1 transcription factor
CTGF Connective tissue growth factor
DG Diacylglycerol
Edg3 Endothelial differentiation gene subfamily 3 receptor, see S1P3
ERK Extracellular-signal-regulated kinase
G_{12/13}, G_{q/11}, Gᵢ Subclasses of guanine nucleotide-binding proteins
IOP Intraocular pressure
IP3 Inositol triphosphate
LPA Lysophosphatidic acid
PAI-1 Plasminogen activator inhibitor 1
PKC Protein kinase C
PLC Phospholipase C
PLD Phospholipase D
Raf Protein kinase raf-1
Ras Small GTP-binding protein
Rho Small GTP-binding protein
S1P Sphingosine-1-phosphate
S1P3 or S1PR3 Sphingosine-1-phosphate receptor 3
Smad-1, -2, -3 Receptor regulated Smad transcription factors
Smad-4 Common partner (Co-) Smad transcription factor
TGFβ Transforming growth factor β
TGFβR, TβRI, TβRII, Transforming growth factor β receptor, -receptor type I, -receptor type II

### SUMMARY OF THE INVENTION

The present invention addresses the above-cited problems in the art and provides a method for attenuating Smad signaling in an eye of a subject by providing antagonists of the S1P-3 receptor. A method of attenuating Smad signaling in an eye of a subject comprises administering to the subject a composition comprising an effective amount of an antagonist of endothelial differentiation gene subfamily 3 receptor or a pharmaceutically acceptable salt thereof, and a pharmaceutically acceptable carrier. Smad signaling in the eye of the subject is attenuated thereby. The subject may have a Smad signaling-associated ocular disorder resulting in inappropriate connective tissue growth factor accumulation or may be at risk of developing such an ocular disorder. The Smad signaling-associated ocular disorder may be ocular hypertension, glaucoma, glaucomatous retinopathy, optic neuropathy, macular degeneration, diabetic retinopathy, choroidal neovascularization, proliferative vitreoretinopathy or ocular wound healing, for example.

The antagonist of endothelial differentiation gene subfamily 3 receptor decreases natural ligand binding to the receptor. The antagonist may comprise an analog of the natural ligand of the receptor, sphingosine-1-phosphate. The antagonist may be a substituted thiazolidine, a substituted thiazinane, or a S1P analog having structure III as cited infra. The antagonist may be a polysulfonated naphthylurea such as suramin, an antibody having binding affinity and specificity for the S1P3 receptor, a biologically active fragment thereof, or a peptide or peptidomimetic having binding affinity and specificity for the receptor.

Another embodiment of the invention is a method of treating a Smad signaling-associated ocular disorder associated with an inappropriate connective tissue growth factor accumulation in a subject in need thereof. The method comprises administering to the subject a composition comprising an effective amount of an antagonist of endothelial differentiation gene subfamily 3 receptor or a pharmaceutically acceptable salt thereof, and a pharmaceutically acceptable carrier. The Smad signaling-associated ocular disorder is treated thereby.

In one embodiment of the invention, a method of treating glaucoma in a subject is provided. The method comprises administering to the subject a composition comprising an effective amount of an antagonist of endothelial differentiation gene subfamily 3 receptor or a pharmaceutically acceptable salt thereof, and a pharmaceutically acceptable carrier, wherein the glaucoma is treated thereby.

In another embodiment of the present invention a method of treating glaucomatous retinopathy, optic neuropathy, macular degeneration, diabetic retinopathy, choroidal neovascularization, proliferative vitreoretinopathy or ocular wound healing in a subject is provided. The method comprises administering to the subject a composition comprising an effective amount of an antagonist of endothelial differentiation gene subfamily 3 receptor or a pharmaceutically acceptable salt thereof, and a pharmaceutically acceptable carrier. The glaucomatous retinopathy, optic neuropathy, macular degeneration, diabetic retinopathy, choroidal neovascularization, proliferative vitreoretinopathy or ocular wound healing is treated thereby.

### BRIEF DESCRIPTION OF THE DRAWINGS

FIG. 1 provides a schematic showing signal transduction involving S1P and Smad, and involving TGF-β and Smad; S1P-1, -2, -3, S1P receptors; TGFβR, TGF-β receptor types 1 and 2 (adapted from Xin et al., JBC, Vol. 279(34):35255-35262, 2004; Blom, et al., Matrix Biology, Vol. 21:473-482, 2002; Takuwa, Y., Biochim Biophys Acta., Vol. 1582:112-120, 2002; Pyne et al., Biochem J, Vol. 349:385-402, 2000; and Xu et al., Acta Pharmacol Sin., Vol. 25:849-854, 2004).

FIG. 2A and FIG. 2B. Human trabecular meshwork cell cultures were treated with (open circles) or without (closed circles) the Edg3 receptor subtype antagonist CAY10444 in the presence of various amounts of the endogenous Edg receptor agonist S1P (FIG. 2A) or in the presence of various amounts of FTY720, a structural analog of SIP (FIG. 2B). Twenty-four hours later, the levels of the secreted PAI-1 protein were then determined by ELISA of supernatant aliquots from the treated cultures as cited in Example 2.

### DETAILED DESCRIPTION OF THE INVENTION

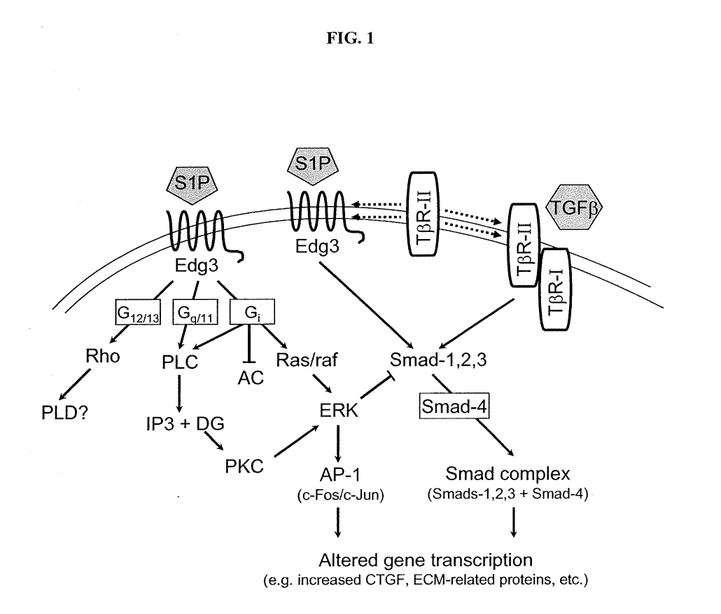

S1P-3 (Edg-3) receptors belong to a family of G-protein coupled receptors for which either LPA or S1P are endogenous ligands. LPA is a ligand for the Edg-2, -4, and -7 receptors and S1P is a ligand for the Edg-1, -3, -5, -6, and -8 receptors. The Edg receptors have been renamed S1P receptors by the International Union of Pharmacology (Chun et al., Pharmacol Rev, Vol. 54:265-269, 2002. Therefore, as used herein, the term “Edg receptor” is synonymous with the term “S1P receptor.” FIG. 1 provides a schematic of a signal transduction relationship between S1P receptors and the regulatory target Smad, and between TGFβ receptors and the same regulatory target Smad. Smad is activated by phosphorylation and complexes with Smad 4 to yield a heteromeric complex which enters the nucleus where the complex, together with other transcription factors, activates gene transcription, such as transcription of the gene encoding CTGF.

Significantly higher levels of TGFβ2 isoform has been found in aqueous humor collected from glaucomatous human eyes as compared to “normal” eyes (Tripathi et al., Exp Eye Res, Vol. 59(6):723-727, 1994; Inatani et al., Graefes Arch Clin Exp Opthalmol, Vol. 239(2):109-113, 2001; Picht et al., Graefes Arch Clin Exp Opthalmol, Vol. 239(3):199-207, 2001; Ochiai et al., Jpn J Opthalmol, Vol. 46(3):249-253, 2002). Furthermore, TGFβ2 is able to provoke substantial increases in IOP in a perfused human anterior segment model (Fleenor et al., Invest Opthalmol V is Sci, Vol. 47(1):226-234, 2006). Therefore, TGFβ, in particular TGFβ2, appears to have a causative role in IOP related disorders such as glaucoma.

The S1P-3 receptors appear to activate Smad signaling pathways in renal mesangial cells (Xin et al., Br J Pharmacol, Vol. 147:164-174, 2006). In addition, Smad proteins are known to mediate the canonical signaling pathways activated by members of the TGF superfamily, including that of TGF-β (as shown by FIG. 1). Therefore, S1P-3-induced activation of Smad protein signaling appears to mimic some of the cellular responses known to be regulated by TGFβ. Further, both TGFβ and S1P are known to increase the expression of CTGF (Xin et al., 2004 Id., Katsuma et al., FEBS Letters, Vol. 579:2576-2582, 2005), a protein that appears to be a key player in the glaucoma process (International Patent Application No. PCT/US2003/012521 to Fleenor et al., published Nov. 13, 2003 as WO 03/092584 and assigned to Alcon, Inc.).

Selective modulation of the TGFβ/S1P3 signaling pathway is desired since TGFβ has a positive role as well as a negative role in tissue. Positive roles include, for example, TGFβ as an anti-inflammatory agent, as an immunosuppressive agent, and as a promoter of migration and homing of T cells. Such selective modulation is provided herein.

The present inventors provide herein antagonists for ocular S1P3 receptors that result in decreased signaling through the Smad receptors, thereby decreasing downstream CTGF accumulation. Modulation of the Smad downstream pathway as provided herein results in a decrease of the negative aspects of TGFβ signaling, while leaving positive signaling effects of TGFβ substantially unaffected. Another embodiment of the invention provides a method of antagonizing S1P3 receptor binding thereby interfering with the S1P3 downstream signaling cascade, and particularly interfering with Smad signaling, for the treatment of ocular disorders in which Smad protein signaling results in inappropriate connective tissue growth factor accumulation.

Antagonists of endothelial differentiation gene subfamily 3 receptor (EDG-3, S1P-3): Antagonists of the S1P-3 receptor include agents that attenuate binding affinity or specificity between the S1P-3 receptor and its natural ligand, S1P. The antagonist may be a S1P analog. Antagonists may be a substituted thiazolidine particularly an alkyl-substituted thiazolidine or an arylalkyl-substituted thiazolidine, a substituted thiazinane particularly an alkyl-substituted thiazinane, a polysulfonated naphthylurea such as suramin (most commonly available as the hexasodium salt), or a S1P analog having structure III as cited infra; an antibody, biologically active antibody fragment thereof, peptide or a peptidomimetic having binding specificity and affinity for the S1P3 receptor; or a pharmaceutically acceptable salt of an antagonist. Antagonist agents as set forth herein may be a racemic mixture, a diastereomer or an enantiomer.

A “pharmaceutically acceptable salt of an antagonist” is a salt of an antagonist that retains the S1P3 receptor antagonistic activity and is acceptable by the human body. Salts may be acid or base salts since antagonists herein may have amino or carboxy substituents.

A substituted thiazolidine has structure I:

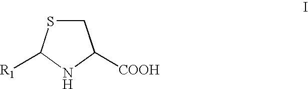

wherein R₁ is C₆-C₁₃ alkyl, or alkyl-substituted aryl where the substitution is C₅-C₉ alkyl. In one embodiment of the invention, the antagonist has structure I where R₁ is C₁₀ alkyl or C₁₁ alkyl, (2-alkylthiazolidine-4-carboxylic acid where the alkyl is C₁₀ or C₁₁). When R₁ is C₁₁ alkyl, the antagonist is CAY10444 available commercially from Cayman Chemical (Ann Arbor, Mich.). In another embodiment of the invention, the antagonist has structure I where R₁ is alkyl-substituted phenyl and the substitution on the phenyl ring is m- or p- C₇-alkyl i.e., (2-(m- or p-heptylphenyl)thiazolidine-4-carboxylic acid).

In one embodiment of the invention, the antagonist of S1P3 has structure II:

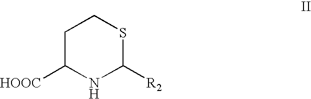

where R₂ is C₉-C₁₃ alkyl.

In another embodiment of the invention, the antagonist of S1P3 has structure III:

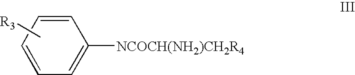

where R₃ is o- or m- C₅-C₈ alkyl; and R₄ is phosphate, phosphate analog, phosphonate, or sulfate. As used herein “phosphate analog” includes the terms phosphoro-thioates, -dithioates, -selenoates, -diselenoates, -anilothioates, -anilidates, -amidates, or boron phosphates, for example.

Further compounds active in S1P3 signaling are described in U.S. Patent Application Publication No. 2005/0222422 to Lynch et al., published Oct. 6, 2005, incorporated by reference herein, and Koide et al., J Med Chem, Vol. 45:4629-4638, 2002.

An assay for identifying further antagonists of S1P3 receptor uses a competitive binding assay which may comprise combining a candidate antagonist, S1P, a S1P3 receptor and a kinase having activity for activated S1P3 receptor and measuring the amount of phosphorylated S1P3 receptor obtained. The result is compared with the amount of phosphorylated S1P3 receptor obtained from the same assay in the absence of the candidate antagonist. The candidate antagonist has antagonist activity when the level of phosphorylated S1P3 receptor is lower than when the candidate is not present. Further assays may include assays for inhibition of receptor specific antibody binding by a candidate antagonist, reduced accumulation of CTGF mRNA by a candidate antagonist, or reduced accumulation of CTGF protein by a candidate antagonist. U.S. Patent Application Publication No. 2005/0222422 to Lynch et al., published Oct. 6, 2005, previously incorporated by reference, describes a GTP binding assay for measuring SIP activity of SIP mimetics to human SIP receptors.

Substituted thiazolidines and substituted thiazinanes are synthesized using methods known in the art, for example, methods described by Koide et al. (J Med Chem, Vol. 45:4629-4638, 2002). U.S. Patent Application Publication No. 2005/0222422 to Lynch et al. published Oct. 6, 2005, previously incorporated by reference describes synthesis of S1P analog having structure III.

Antibodies having binding specificity and affinity for the S1P3 receptor are available commercially, for example, a mouse monoclonal antibody is available from GENETEX, Inc. (Catalog Number GTX12254, San Antonio, Tex.), a rabbit polyclonal antibody to sphingolipid receptor Edg3/S1P3 is available from Novus Biologics Inc. (Catalog Number NLS 1031, Littleton, Colo.), and the EDG-3 CT antibody is available from Exalpha Biologicals, Inc. (Watertown, Mass.). EDG-3 CT has binding affinity and specificity for the unique C-terminal peptide of human S1P3 receptor.

Antagonism of S1P-3 receptors and resultant inhibition of CTGF accumulation is also inferred in a human or mammal by observing an improvement in an ocular disorder. For example, in age-related macular degeneration a slowing or reversal of vision loss indicates inhibition of CTGF accumulation and, in glaucoma patients, lowered intraocular pressure and a delay or prevention of the onset of symptoms in a subject at risk for developing glaucoma indicates inhibition of CTGF accumulation.

Antagonists of the present invention may be used in combination with other agents for treating ocular disorders where CTGF accumulation or activity is inappropriate such as, for example, agents described by U.S. Published Patent Application No. 2005/0234075 to Fleenor et al., published Oct. 20, 2005, previously incorporated by reference herein.

Mode of administration: The antagonist may be delivered directly to the eye (for example: topical ocular drops or ointments; slow release devices in the cul-de-sac or implanted adjacent to the sclera (transscleral) or within the eye; periocular, conjunctival, sub-Tenons, intracameral, intravitreal, sub-retinal, retrobulbar, or intracanalicular injections) or systemically (for example: oral; intravenous, subcutaneous or intramuscular injections; parenterally, dermal delivery) using techniques well known by those skilled in the art. It is further contemplated that the antagonists of the invention may be formulated in a placement device such as a retinal pellet, intraocular insert, catheter, suppository or an implant device comprising a porous, non-porous, or gelatinous material. Intracameral injection may be through the cornea into the anterior chamber to allow the agent to reach the trabecular meshwork. Intracanalicular injection may be into the venous collector channels draining Schlemm's canal or into Schlemm's canal.

Subject: A subject in need of treatment for an ocular disorder or at risk for developing an ocular disorder is a human or other mammal having a condition or at risk of having a condition associated with Smad activation with inappropriate accumulation of CTGF. Such an ocular disorder may include, for example, hypertension, glaucoma, macular degeneration, diabetic retinopathy, choroidal neovascularization, proliferative vitreoretinopathy, ocular wound healing, and conditions with excessive scarring, with endothelial cell proliferation, or fibroproliferation. Ocular structures associated with such disorders may include the retina, choroid, lens, cornea, trabecular meshwork, rod, cone, ganglia, macula, iris, sclera, aqueous chamber, vitreous chamber, ciliary body, optic disc, papilla, or fovea, for example.

Formulations and Dosage: Pharmaceutical formulations comprise an antagonist, or salt thereof, as set forth herein up to 99% by weight mixed with a physiologically acceptable ophthalmic carrier medium such as water, buffer, saline, glycine, hyaluronic acid, mannitol, and the like. Examples of possible formulations embodied by aspects of the invention are as follows.

| Compound | Amount in Weight % |
|---|---|
| S1P-3 receptor antagonist | up to 99; 0.1-99; 0.1-50; |
| | 0.5-10.0; 0.01-5.0; 0.01-2.0; |
| | 0.02-2.0; 0.1-1.0; 0.5-2.0 |
| Hydroxypropylmethylcellulose | 0.5 |
| Sodium chloride | .8 |
| Benzalkonium Chloride | 0.01 |
| EDTA | 0.01 |
| NaOH/HCl | qs pH 7.4 |
| Purified water | qs 100 mL |

| Compound | Amount in Weight % |
|---|---|
| S1P-3 receptor antagonist | up to 99; 0.1-99; 0.1-50; |
| | 0.5-10.0; 0.01-5.0; 0.01-2.0; |
| | 0.02-2.0; 0.1-1.0; 0.5-2.0; |
| | 0.00005-0.5; 0.0003-0.3; |
| | 0.0005-0.03; 0.001 |
| Phosphate Buffered Saline | 1.0 |
| Benzalkonium Chloride | 0.01 |
| Polysorbate 80 | 0.5 |
| Purified water | q.s. to 100% |

| Compound | Amount in Weight % |
|---|---|
| S1P-3 receptor antagonist | up to 99; 0.1-99; 0.1-50; |
| | 0.5-10.0; 0.01-5.0; 0.01-2.0; |
| | 0.02-2.0; 0.1-1.0; 0.5-2.0; |
| | 0.001 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water | q.s. to 100% |

| Compound | Amount in Weight % |
|---|---|
| S1P-3 receptor antagonist | up to 99; 0.1-99; 0.1-50; |
| | 0.5-10.0; 0.01-5.0; 0.01-2.0; |
| | 0.02-2.0; 0.1-1.0; 0.5-2.0; |
| | 0.0005 |
| Phosphate Buffered Saline | 1.0 |
| Hydroxypropyl-β-cyclodextrin | 4.0 |
| Purified water | q.s. to 100% |

In a further embodiment, the ophthalmic compositions are formulated to provide for an intraocular concentration of about 0.1-100 nanomolar (nM) or, in a further embodiment, 1-10 nM of the antagonist. Topical compositions are delivered to the surface of the eye one to four times per day according to the routine discretion of a skilled clinician. The pH of the formulation should be 4-9, or 4.5 to 7.4. Systemic formulations may contain about 10 to 1000 mg of the antagonist.

An “effective amount” refers to that amount of S1P-3 receptor antagonist that is able to disrupt binding between the S1P-3 receptor and Smad. Such disruption leads to lowered Smad activity, lowered CTGF gene transcription, lowered CTGF protein accumulation and resultant lessening of symptoms in ocular disorders in a subject. Such disruption delays or prevents the onset of symptoms in a subject at risk for developing ocular disorders as set forth herein. The effective amount of a formulation may depend on factors such as the age, race, and sex of the subject, or the severity of the ocular condition, for example. In one embodiment, the antagonist is delivered topically to the eye and reaches the trabecular meshwork, retina or optic nerve head at a therapeutic dose thereby ameliorating the ocular disease process.

Acceptable carriers: An ophthalmically acceptable carrier refers to those carriers that cause at most, little to no ocular irritation, provide suitable preservation if needed, and deliver one or more S1P-3 antagonists of the present invention in a homogenous dosage. For ophthalmic delivery, a S1P-3 antagonist may be combined with opthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the antagonist in a physiologically acceptable isotonic aqueous buffer. Further, the ophthalmic solution may include an opthalmologically acceptable surfactant to assist in dissolving the antagonist. Viscosity building agents, such as hydroxymethylcellulose, hydroxyethylcellulose, methylcellulose, polyvinylpyrrolidone, or the like, may be added to the compositions of the present invention to improve the retention of the compound.

In order to prepare a sterile ophthalmic ointment formulation, the S1P-3 antagonist is combined with a preservative in an appropriate vehicle, such as mineral oil, liquid lanolin, or white petrolatum. Sterile ophthalmic gel formulations may be prepared by suspending the S1P-3 antagonist in a hydrophilic base prepared from the combination of, for example, CARBOPOL®-940 (BF Goodrich, Charlotte, N.C.), or the like, according to methods known in the art for other ophthalmic formulations. VISCOAT® (Alcon Laboratories, Inc., Fort Worth, Tex.) may be used for intraocular injection, for example. Other compositions of the present invention may contain penetration enhancing agents such as cremophor and TWEEN® 80 (polyoxyethylene sorbitan monolaureate, Sigma Aldrich, St. Louis, Mo.), in the event the S1P-3 antagonists are less penetrating in the eye.

Kits: Embodiments of the present invention provide a kit that includes antagonists for attenuating S1P3 receptor signaling in a cell. The kit contains in close confinement one or more containers containing an antagonist of the present invention, a pharmaceutically acceptable carrier and, optionally, printed instructions for use.

### Example 1

### Inhibition of S1P-Stimulated CTGF Gene Expression

The effect of Edg3 receptor antagonism on CTGF gene expression in cultured human trabecular meshwork cells can be determined as follows. Transformed or non-transformed human TM cell cultures (Pang et al., Curr Eye Res, Vol. 13:51-63, 1994; Steely et al., Invest Opthalmol Vis Sci, Vol. 33:2242-2250, 1992; Wilson et al., Curr Eye Res, Vol. 12:783-793, 1993; Stamer et al., Curr Eye Res, Vol. 14:611-617, 1995) are treated with or without a stimulatory amount of sphingosine-1-phosphate (S1P) and with or without Edg3 receptor antagonists for a specified period of time. Separate cultures are also treated with the requisite diluent vehicle(s) used in order to serve as controls. Total RNA is then isolated from the TM cells using Qiagen RNeasy 96 system according to the manufacturer's instructions (Qiagen).

Differential expression of CTGF after cell treatment is verified by quantitative real-time RT-PCR (QRT-PCR) using an ABI Prism® 7700 Sequence Detection System (Applied Biosystems) essentially as previously described (Shepard et al., IOVS, Vol. 42:3173, 2001). Primers for CTGF amplification were designed using Primer Express software (Applied Biosystems) to anneal to adjacent exons of Genbank accession #NM 001901.1 as set forth in U.S. Published Patent Application No. 20050234075 to Fleenor et al., published Oct. 20, 2005, U.S. Ser. No. 10/510,585, filed Oct. 8, 2004, (incorporated by reference herein) to generate a 76-bp amplicon. Amplification of CTGF is normalized to 18S ribosomal RNA expression using primers designed to the 18S rRNA gene (GenBank accession #X03205) as cited by U.S. Published Patent Application No. 20050234075 to Fleenor et al. Id., to generate a 69-bp amplicon. CTGF QRT-PCR is performed in multiplex with 18S primer/probe sets in a 50 ul final volume consisting of 40 nM 18S or 900 nM CTGF primers; 100 nM 18S probe or 100 nM CTGF; 5 ul RNA; 1× Multiscribe and RNase Inhibitor Mix (ABI); and 1× TaqMan® Universal Mix (ABI). Thermal cycling conditions consist of 48° C. for 30 min and 95° C. for 10 min, followed by 40 cycles at 95° C. for 15 sec and 60° C. for 1 min. Data analysis is performed with SDS software version 1.9.1 (Applied Biosystems) and MS Excel 2002 (Microsoft). Quantification of relative RNA concentrations is done using the delta delta Ct method as described in PE Biosystems User Bulletin #2. Levels of amplified products are expressed as mean±SEM of quadruplicate QRT-PCR assays. Data analysis is performed with SDS software version 1.9.1 (Applied Biosystems) and MS Excel 97 (Microsoft).

### Example 2

### Inhibition of SIP-Stimulated Change in Expression of Extracellular Matrix-Related Proteins

The effect of Edg3 receptor antagonism on expression of extracellular matrix-related proteins by cultured human trabecular meshwork cells is determined as follows. Human TM cell cultures are split into replicate and/or experimental and/or control groups to which are then added control solutions or experimental solutions comprising diluent vehicle(s) (as controls) and/or S1P (as stimulatory agent) and/or Edg3 receptor antagonists. Levels of extracellular matrix-related proteins, such as fibronectin, plasminogen activator inhibitor I (PAI-1), collagens, fibrillin, vitronectin, laminin, thrombospondin I, proteoglycans, or integrins, are then measured in each cell culture group via standard enzyme-linked immunoabsorbent assays (ELISA). Such assays are well-known to those skilled in the art and are sensitive immunoassays which utilize an enzyme linked to an antibody or antigen as a marker for the detection of a specific protein. By these means, levels of various extracellular matrix-related proteins can then be compared between the groups in order to determine the effect of experimental solutions.

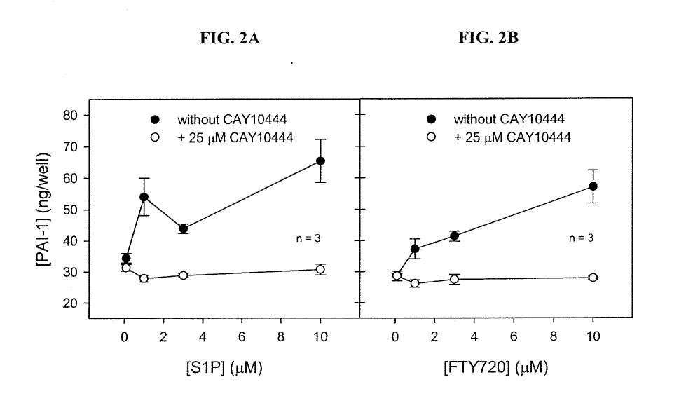

An example of the effect of Edg3 receptor antagonism on PAI-1 levels in supernatants from treated human TM cell cultures is shown in FIG. 2A and FIG. 2B. For these studies, human TM cell cultures were treated with or without the Edg3 receptor subtype antagonist CAY10444 in the presence of various amounts of the endogenous Edg receptor agonist S1P and/or in the presence of various amounts of FTY720, a structural analog of S1P. Twenty-four hours later, the levels of the secreted PAI-1 protein were then determined by ELISA of supernatant aliquots from the treated cultures. It is apparent from these data that the effect of both agonists was potently and efficaciously antagonized by CAY10444 (data represent mean and SEM).

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

As used herein and unless otherwise indicated, the terms “a” and “an” are taken to mean “one”, “at least one” or “one or more.”

1-35. (canceled)

36. A method for reducing levels of extracellular matrix-related proteins in the trabecular meshwork of an eye of a subject, thereby lowering intraocular pressure, comprising:
administering to the subject a topical composition comprising:
an effective amount of an antagonist of endothelial differentiation gene subfamily 3 receptor or a pharmaceutically acceptable salt thereof; and
a pharmaceutically acceptable carrier;
wherein levels of extracellular matrix-related proteins in the trabecular meshwork of the eye are reduced and intraocular pressure is lowered thereby; and
wherein the antagonist is of structure I: 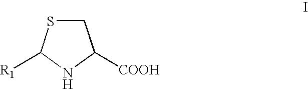
wherein R₁ is C₆-C₁₃ alkyl, or alkyl-substituted aryl where the aryl substitution is C₅-C₉ alkyl.

37. A method according to claim 36 wherein the subject has a Smad signaling-associated ocular disorder with inappropriate connective tissue growth factor accumulation.

38. A method according to claim 36 wherein the subject is at risk of developing a Smad signaling-associated ocular disorder with inappropriate accumulation of connective tissue growth factor.

39. A method according to claim 37 wherein the Smad signaling-associated ocular disorder is ocular hypertension, glaucoma, glaucomatous retinopathy, optic neuropathy, macular degeneration, diabetic retinopathy, choroidal neovascularization, proliferative vitreoretinopathy or ocular wound healing.

40. A method according to claim 36 wherein the concentration of the antagonist in the composition is from 0.01% to 2%.

41. A method according to claim 36 wherein the composition is administered via a topical, intracameral, intravitreal, transcleral, or an implant route.

42. A method of treating glaucoma in a subject, comprising:
administering to the subject a topical composition comprising:
an effective amount of an antagonist of endothelial differentiation gene subfamily 3 receptor or a pharmaceutically acceptable salt thereof; and
a pharmaceutically acceptable carrier;
wherein levels of extracellular matrix-related proteins in the trabecular meshwork of the eye are reduced and intraocular pressure is lowered thereby; and
wherein the antagonist is of structure I: 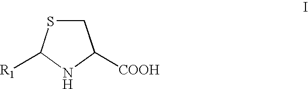
wherein R₁ is C₆-C₁₃ alkyl, or alkyl-substituted aryl where the aryl substitution is C₅-C₉ alkyl.

43. A method according to claim 42 wherein the concentration of the antagonist in the composition is from 0.01% to 2%.

44. A method according to claim 42 wherein the composition is administered via a topical, intracameral, intravitreal, transcleral, or an implant route.
